(19) United States
(12) Patent Application Publication
(10) Pub. No.: US 2025/0029729 A1
(43) Pub. Date: Jan. 23, 2025

(54) MACHINE LEARNING-BASED RISK-CLASSIFICATION OF ENDOMERIAL CANCER

(71) Applicant: Northwestern University, Evanston, IL (US)

(72) Inventors: Shuhua Zheng, Chicago, IL (US); Eric Donald Donnelly, Chicago, IL (US); Jonathan B. Strauss, Chicago, IL (US)

(21) Appl. No.: 18/780,061

(22) Filed: Jul. 22, 2024

Related U.S. Application Data
(60) Provisional application No. 63/514,742, filed on Jul. 20, 2023.

(51) Int. Cl. G16H 50/30; G16H 10/60

(52) U.S. Cl. CPC G16H 50/30; G16H 10/60

(57) ABSTRACT

Endometrial cancer is classified and/or risk stratified using a suitably trained machine learning model. Risk classification of endometrial cancer is provided using a machine learning-based analysis of patient health data, such as clinicopathologic data, molecular data, and the like. Risk assessment is optimized for endometrial cancer, including risk of nodal involvement, distant metastasis, disease progression, and overall survival.

### CROSS-REFERENCE TO RELATED APPLICATIONS

This application claims the benefit of U.S. Provisional Patent Application Ser. No. 63/514,742, filed on Jul. 20, 2023, and entitled “MACHINE LEARNING-BASED RISK-CLASSIFICATION OF ENDOMETRIAL CANCER,” which is herein incorporated by reference in its entirety.

### BACKGROUND

Risk stratification of endometrial carcinoma (EC) on the basis of historical clinical pathologic features are useful but imperfect owing to limitations in both inter-rater reliability of scoring and ultimate prognostic value. The Cancer Genome Atlas (TCGA) project identified four molecular subgroups of EC, including DNA polymerase epsilon (POLE) ultramutated, mismatch repair deficiency (dMMR), copy-number (CN)-low, and CN-high. This molecular classification system has demonstrated a high degree of reproducibility and clinical relevance.

Based on analyses of biobank data from European clinical trials Post-Operative Radiation Therapy in Endometrial Carcinoma (PORTEC) 1 and 2, the classification system was later simplified by the ProMisE (Proactive Molecular Risk Classifier for Endometrial Cancer) and Leiden/TransPORTEC molecular classification systems. Both ProMisE and Leiden/TransPORTEC systems proposed the use of p53 immunohistochemistry (IHC) staining as a surrogate for CN-high group in order to avoid costly genome-wide sequencing, improving clinical feasibility. These molecular classification systems are both prognostic for outcomes and predictive of treatment benefit.

Subgroup analysis of PORTEC 3 showed that the addition of paclitaxel/carboplatin chemotherapy to radiotherapy (RT) improved relapse-free survival (RFS) in TP53-mutated subgroup, with a trend for benefit in the No Specific Molecular Profile (NSMP) group, whereas chemotherapy yielded no benefit in the POLE or dMMR groups. Similarly, the Gynecologic Oncology Group (GOG)-86P trial of advanced-stage EC cases showed the addition of bevacizumab to frontline chemotherapy regimens improved survival only in those carrying TP53 mutations.

However, using TP53 as a surrogate of the CN-high molecular subgroup based on a European population represented in the PORTEC1/2 biobank may imperfectly reflect the relationship between those variables in other populations. Next-generation sequence (NGS) has identified TP53 mutations in the tumors of 70% percent of Black or African American (BOAA) woman as compared to about 50% in non-BOAA patients with EC. Similarly, a higher proportion of BOAA patients with EC exhibit grade 3 and serous histology and CN-high molecular features, and are less likely to have PTEN mutations as compared with Caucasians. Meanwhile, both PORTEC and GOG 99 trials identified age at EC diagnosis as an independent risk factor in risk classification for adjuvant chemoradiotherapy treatment planning. The utility of a molecular classification system derived from a European population requires validation in demographically diverse populations.

In addition to molecular subgroups, other clinicopathologic variables contribute to prognosis and treatment decisions including histologic subtype, age-at-diagnosis, and the presence of (and extent of) lymphovascular invasion (LVSI). These clinicopathologic variables are not fully captured by a molecular-based classification system. Additionally, increasing age is consistently associated with worse disease-specific outcome in EC patients although the underlying cause of this relationship is not clear. The distribution of molecular subtypes by age may vary.

### SUMMARY OF THE DISCLOSURE

It is an aspect of the present disclosure to provide a method for risk stratifying a patient for endometrial cancer using machine learning. The method includes accessing patient health data for a patient with a computer system and accessing a machine learning model with the computer system. The machine learning model has been trained on training data to generate classified feature data based on features present in a patient's patient health data. The patient health data are applied to the machine learning model, generating an output as classified feature data that indicate at least one of a risk stratification or classification of endometrial cancer in the patient based on features in their patient health data.

### BRIEF DESCRIPTION OF THE DRAWINGS

FIG. 1 a flowchart setting forth the steps of an example method for generating classified feature data from patient health data using a machine learning model, where the classified feature data are indicative of risk and or classification of endometrial cancer.

FIG. 2 is a flowchart setting forth the steps of an example method for training a machine learning model to generate classified feature data (e.g., NU-CATS scores and associated data) from patient health data.

FIG. 3 shows an example workflow in developing and cross-validation of an example NU-CATS classification. Endometrial cancer (EC) patients from the Cancer Genome Atlas (TCGA) Uterine Corpus Endometrial Carcinoma (TCGA-UCEC) (n=596), Memorial Sloan Kettering-Metastatic Events and Tropisms (MSK-MET, n=1,315) were selected. Data preprocessing includes feature selection and target identification. Patients with information regarding required features was selected in the TCGA-UCEC and MSK-MET datasets. Deep learning models were then developed based on MSK-MET datasets, and cross validated for distant metastasis prediction, overall survival (OS) prognosis, and early-stage EC progression in MSK-MET and TCGA-UCEC datasets.

FIGS. 4A-4E show an example of artificial neural network (ANN) feature selection, target identification and ANN construction. FIG. 4A shows a correlation study of features and phenotypes representing disease progressions. Black square: correlation between features; Red square: correlation between intra-abdominal progression (IAP) and features. FIG. 4B shows an OS analysis of patients with (W/) IAP (n=463) and without (W/O) IAP (n=807) (p<0.0001). FIG. 4C shows distant metastasis pattern of patients W/IAP and W/O IAP. Yes: W/IAP; No: W/O IAP. True: positive for metastasis; False: negative for metastasis. FIG. 4D shows a loss of function and receiver operating characteristic (ROC) curve analysis (AUC=0.76). FIG. 4E shows a schematic overview of an example ANN architecture that was the best performing ANN in an example study, where the ANN has a ‘5-6-4-2-1’ structure.

FIGS. 5A-5F show an example NU-CATS validation in disease progression prediction. FIGS. 5A and 5B show risk of EC patients with distant metastasis at specified organs/locations at different NU-CATS ranges (30-45, 45-50, 50-70, and ≥70). FIG. 5C shows a total amount of distant metastasis at different NU-CATS ranges. FIGS. 5D and 5E show progression free survival (PFI) of Stage I and II EC patients classified based on NU-CATS Score and TransPORTEC classification system. ns: non-significant. FIG. 5F shows a risk of positive pelvic lymph nodes (LNs) and para-aortic LNs at different NU-CATS ranges.

FIGS. 6A-6H show an example of impact of race on EC survival. FIG. 6A shows an OS analysis of EC patients with metastatic lesions (n=107, n=643 for BOAA and Caucasian, respectively, p=0.003). FIG. 6B shows an OS analysis for those without metastatic lesions (n=24, n=313 for BOAA and Caucasian, respectively, p=0.0005). FIG. 6C shows an OS analysis for those W/TP53 mutations (n=97, n=389 for BOAA and Caucasian, respectively, p=0.012). FIG. 6D shows an OS analysis for BOAA patients (n=95) as compared with Caucasian (n=303) in the subgroup analysis of UCS and USC histology. FIG. 6E shows an example volcano plot comparing the genetic discrepancy between BOAA and Caucasian EC patients. FIG. 6F shows the quantification of mutation counts between BOAA and Caucasian EC patients. FIG. 6G shows a fraction genome altered (FGA) score for BOAA and Caucasian EC patients. (****: p<0.0001). FIG. 6H illustrates that BOAA patients have more frequently exhibit mutations in TP53 (74.6% vs. 41.0. %), and less frequently exhibit mutations in PTEN (23.9% vs. 50.7%, p<0.001), PIK3CA (33.6% vs. 49.2%, p<0.001), and ARID1A (16.4% vs. 43.5%, p<0.001).

FIGS. 7A-7E show an example of impact of age on EC survival. FIGS. 7A and 7B show patients in general, and BOAA patients, from MSK-MET were grouped based on age at surgery into 3 groups i.e., age between 20-60, 60-70, and 70-90. Percentage of genetic mutations of corresponding genes in each age group were presented. FIG. 7C shows an OS analysis of EC patients on corresponding age groups (p<0.0001). FIG. 7D shows a percentage of histology subtypes in age groups. USC: uterine serous carcinoma; UEC: uterine endometroid carcinoma; UCS: uterine carcinosarcoma (p<0.0001). FIG. 7E shows percentages of patients with distant metastasis. True: positive for metastasis; False: negative for metastasis (p<0.0001).

FIGS. 8A-8D show an example of NU-CATS validation in OS prediction. FIGS. 8A and 8B shows OS analyses of subgroups of EC patients classified based on NU-CATS ranges (NU-CATS≤50, 50<NU-CATS≤65, NU-CATS≥65) in MSK-MET (FIG. 8A) and TCGA-UCEC (FIG. 8B) datasets (p<0.0001). FIGS. 8C and 8D show OS analyses of subgroups of EC patients classified based on TransPORTEC risk classification system for MSK-MET and TCGA-UCEC datasets. MSI: Microsatellite instability; NSMP: no specific molecular profile (p<0.0001).

FIGS. 9A-9E show an example of NU-CATS for BOAA EC patients prognosis and model comparison with PORTEC 4a study design. FIGS. 9A and 9B show OS analysis for BOAA (FIG. 9A) and Caucasian (FIG. 9B) EC patients in subgroups classified by NU-CATS score ranges. *: p<0.05, **: p<0.01. FIG. 9C shows an OS analysis for BOAA in subgroups classified TransPORTEC system. ns: p-value non-significant. FIG. 9D shows a schematic overview of the NU-CATS in classifying EC patients into different risk groups. FIG. 9E shows an example schematic overview of the risk classification used in the PORTEC 4a trial.

FIG. 10 is a block diagram of an example system for generating classified feature data such as NU-CATS risk scores and associated data.

FIG. 11 is a block diagram of example components that can implement the system of FIG. 10.

### DETAILED DESCRIPTION

Described here are systems and methods for classifying and/or risk stratifying endometrial cancer using a suitably trained machine learning model. In general, the systems and methods provide risk classification of endometrial cancer using a machine learning-based analysis of patient health data, such as clinicopathologic data, molecular data, and the like. In this way, a machine learning model that incorporates clinical and molecular risk factors is used to optimize risk assessment for endometrial cancer, including risk of nodal involvement, distant metastasis, disease progression, and overall survival. Advantageously, the disclosed systems and methods provide a machine learning model that is capable of generating classified feature data that indicate prognosis of endometrial cancer, such as endometrial carcinoma.

The classified feature data generated using the systems and methods described in the present disclosure may be referred to, in some examples, as a machine learning-based New Unified classifiCATion Score (NU-CATS). In some implementations, the machine learning model may take the following as input data: age, race, histology, mismatch repair status, and TP53 mutation status. Advantageously, a NU-CATS score based on these inputs has demonstrated 75% accuracy in prognosticating intra-abdominal progression. A higher NU-CATS score may be associated with an increasing risk of having positive pelvic or para-aortic lymph nodes and distant metastasis. Advantageously, in an example study, NU-CATS was shown to outperform the Leiden/TransPORTEC model for estimating risk of FIGO Stage I/II disease progression and survival in BOAA EC patients.

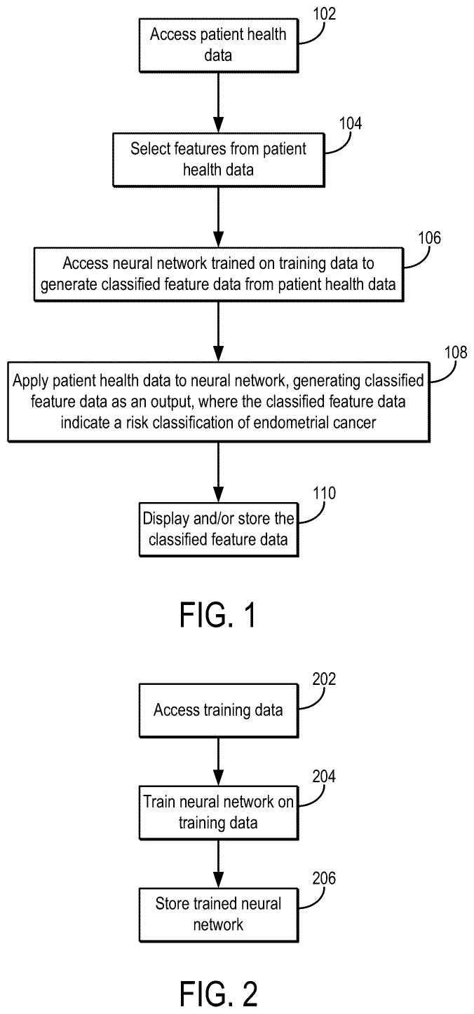

Referring now to FIG. 1, a flowchart is illustrated as setting forth the steps of an example method for generating classified feature data using a suitably trained neural network or other machine learning algorithm. As will be described, the neural network or other machine learning algorithm takes patient health data as input data and generates classified feature data as output data. As an example, the classified feature data can be indicative of a risk score for developing endometrial cancer, classifications of endometrial cancer types or subtypes, and the like. In some instances, the risk scores can be predictive and/or prognostic, such as by indicating risk of nodal involvement, distant metastasis, disease progression, and overall survival.

The method includes accessing patient health data with a computer system, as indicated at step 102. Accessing the patient health data may include retrieving such data from a memory or other suitable data storage device or medium.

The patient health data may include data stored in, retrieved from, extracted from, or otherwise derived from the patient's electronic medical record (“EMR”) and/or electronic health record (“EHR”). The patient health data can include unstructured text, questionnaire response data, clinical laboratory data, histopathology data, genetic sequencing, medical imaging, and other such clinical data types. Examples of clinical laboratory data and/or histopathology data can include genetic testing and laboratory information, such as performance scores, lab tests, pathology results, prognostic indicators, date of genetic testing, testing method used, and so on.

The patient health data may also include other clinical severity of illness scales and observations commonly used and documented in the medical record.

Features derived from structured, curated, and/or EHR data may include clinical features such as diagnoses; symptoms; therapies; outcomes; patient demographics, such as patient name, date of birth, age, gender, race, and/or ethnicity; diagnosis dates for cancer, illness, disease, or other physical or mental conditions; personal medical history; family medical history; clinical diagnoses, such as date of initial diagnosis, date of metastatic diagnosis, cancer staging, tumor characterization, and tissue of origin; and the like. Additionally, the patient health data may also include features such as treatments and outcomes, such as line of therapy, therapy groups, clinical trials, medications prescribed or taken, surgeries, radiotherapy, imaging, adverse effects, and associated outcomes.

Patient health data can include a set of clinical features associated with information derived from clinical records of a patient, which can include records from family members of the patient. These clinical features and data may be abstracted from unstructured clinical documents, EHR, or other sources of patient history. Such data may include patient symptoms, diagnosis, treatments, medications, therapies, responses to treatments, laboratory testing results, medical history, geographic locations of each, demographics, or other features of the patient which may be found in the patient's EHR.

The patient health data may also include molecular data or other omics data. As one example, molecular data may include TP53, POLE, PTEN and CTNNB1 mutations, MMR status, and copy number variations (CNVs).

In some instances, the patient health data can additionally or alternatively include one or more types of omics data, such as genomics data, proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

As a non-limiting example, epigenomics data may include data associated with information derived from DNA modifications that are not changes to the DNA sequence and regulate the gene expression. These modifications can be a result of environmental factors based on what the patient may breathe, eat, or drink. These features may include DNA methylation, histone modification, or other factors which deactivate a gene or cause alterations to gene function without altering the sequence of nucleotides in the gene.

Microbiomics data may include, for example, data derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

Metabolomics data may include molecules obtained from the blood, cerebrospinal fluid (CSF), and body compartments in patients.

Proteomics data may include data associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another; or modifications to the protein after translation from the RNA such as phosphorylation, ubiquitination, methylation, acetylation, glycosylation, oxidation, or nitrosylation.

Genomics data may include genomic information that can be, or have been, correlated with the symptoms and medication effect, tolerance, and/or side effect information that may be received from a patient as responses to a questionnaire and stored as questionnaire response and/or phenotypic data. As a non-limiting example, genomics data can be extracted from blood or saliva samples collected from individuals who have also completed one or more questionnaires such that corresponding questionnaire response data is available for the individuals. A deep phenotypic characterization of these individuals can be assembled. As an example, in one large subset, prospectively determined patterns of treatment response after protocoled titrations in various different drugs from distinct classes of treatments have been assembled. For instance, an analysis of Verapamil, (an L-type calcium channel blocker) using whole exome sequencing (WES) can be completed following genotyping in a confirmatory cohort.

In some instances, patient health data can include medical imaging data, which may include images of the patient obtained with one or more different medical imaging modalities, including magnetic resonance imaging (MRI), computed tomography (CT), x-ray imaging, positron emission tomography (PET), ultrasound, and so on. The medical imaging data may also include parameters or features computed or derived from such images. Medical imaging data may also include digital pathology images, such as H&E slides, IHC slides, and the like. The medical imaging data may also include data and/or information from pathology and radiology reports, which may be ordered by a physician during the course of diagnosis and treatment of various illnesses and diseases.

In some embodiments, the patient health data can include a collection of data and/or features including all of the data types disclosed above. Alternatively, the patient health data may include a selection of fewer data and/or features.

As indicated at step 104, in some embodiments a subset of features that have been identified as having higher importance or relevance to risk stratifying endometrial cancer can be selected from the patient health data. As a non-limiting example, the features may include some or all of age, disease stage, histologic subtype and grades, race, TP53 status, mismatch repair (MMR) status, fraction genome altered (FGA), and mutation counts. In some embodiments, the subset of features can be selected using a machine learning algorithm, such as a decision tree-based method that ranks the importance of features in the patient health data across a large cohort of patients.

A trained neural network (or other suitable machine learning algorithm) is then accessed with the computer system, as indicated at step 106. In general, the neural network is trained, or has been trained, on training data in order to generate classified feature data from patient health data, where the classified feature data are indicative of risk scores and/or classifications of endometrial cancer. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network generates risk scores (e.g., NU-CATS risk scores), the output layer may include a node that corresponds to generating a risk score. When the artificial neural network generates other classified feature data (e.g., classifications), a first node may indicate a first classification (e.g., an endometrial cancer type or subtype), and a second node may indicate a second classification (e.g., a prognosis for whether the patient will develop nodal involvement, distant metastasis, etc.). In some embodiments, the output classified feature data may include both a classification and a risk score and/or probability of a prognostic indication.

The patient health data are then input to the one or more trained neural networks, generating output as classified feature data, as indicated at step 108. For example, the classified feature data may include a risk score. The risk score can provide physicians or other clinicians with a recommendation to consider additional monitoring for subjects whose patient health data indicate the likelihood of the subject suffering from a particular medical condition (e.g., endometrial cancer) and/or developing certain conditions associated with endometrial cancer, such as nodal involvement, distant metastasis, disease progression, etc.

As another example, the classified feature data may indicate the probability for a particular classification (i.e., the probability that the patient health data include patterns, features, or characteristics indicative of detecting, differentiating, and/or determining the severity of endometrial cancer). The probability may be a quantitative score (e.g., a probability percentage or value), or may include classification labels for the relative risk of developing endometrial cancer. For example, the category labels may indicate low, moderate, or high risk for developing endometrial cancer.

Additionally or alternatively, the classified feature data may classify the patient health data as indicating a particular medical condition. In these instances, the classified feature data can differentiate between different medical conditions (e.g., different conditions associated with endometrial cancer, such as nodal involvement, distant metastasis, stages of disease progression, etc.). In still other embodiments, the classified feature data may indicate a severity of endometrial cancer. For example, the classified feature data may include a severity score that quantifies a severity of endometrial cancer.

Additionally or alternatively, the classified feature data can be further processed to generate a risk score for the patient developing endometrial cancer. For instance, a risk score can be calculated by taking the integral numbers of sigmoid activation values in the classified feature data. This risk score, which may be referred to as a NU-CATS score, can also be multiplied by 100 to adjust the scaling presented to a user. In still other embodiments, the neural network can output classified feature data that include a NU-CATS score.

The classified feature data generated by inputting the patient health data to the trained neural network(s) can then be displayed to a user, stored for later use or further processing, or both, as indicated at step 108. In some embodiments the classified feature data can be analyzed by a computer system to generate an order set for follow up examination of the patient. For example, if the classified feature data indicate the patient is at high risk for nodal involvement in endometrial cancer, an order set for further examination associated with determining the extent of nodal involvement can be generated and entered into the EHR system to order the further testing for the patient. Additionally or alternatively, the order set may also include treatment options and/or less invasive orders or suggestions for the patient.

Referring now to FIG. 2, a flowchart is illustrated as setting forth the steps of an example method for training one or more neural networks (or other suitable machine learning algorithms) on training data, such that the one or more neural networks are trained to receive patient health data as input data in order to generate classified feature data as output data, where the classified feature data are indicative of risk scores for developing endometrial cancer and/or classifications of endometrial cancer types or subtypes.

In general, the neural network(s) can implement any number of different neural network architectures. For instance, the neural network(s) could implement a deep neural network, a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on.

The method includes accessing training data with a computer system, as indicated at step 202. Accessing the training data may include retrieving such data from a memory or other suitable data storage device or medium. In general, the training data can include patient health data, such as those types of patient health data described above. In some embodiments, the training data may include patient health data that have been labeled (e.g., labeled as containing patterns, features, or characteristics indicative of endometrial cancer; progressions of endometrial cancer including nodal involvement, distant metastasis, etc.; and the like).

The method can include assembling training data from patient health data using a computer system. This step may include assembling the patient health data into an appropriate data structure on which the neural network or other machine learning algorithm can be trained. Assembling the training data may include assembling patient health data and other relevant data. For instance, assembling the training data may include generating labeled data and including the labeled data in the training data. Labeled data may include patient health data or other relevant data that have been labeled as belonging to, or otherwise being associated with, one or more different classifications or categories. For instance, labeled data may include patient health data that have been labeled as being associated with endometrial cancer, a particular progression of endometrial cancer, etc.

One or more neural networks (or other suitable machine learning algorithms) are trained on the training data, as indicated at step 204. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as classified feature data. The artificial neural network then compares the generated output with the actual output of the training example in order to evaluate the quality of the classified feature data. For instance, the classified feature data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

The one or more trained neural networks are then stored for later use, as indicated at step 206. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

In an example study, the systems and methods described in the present disclosure were implemented to assess patient with potential endometrial cancer.

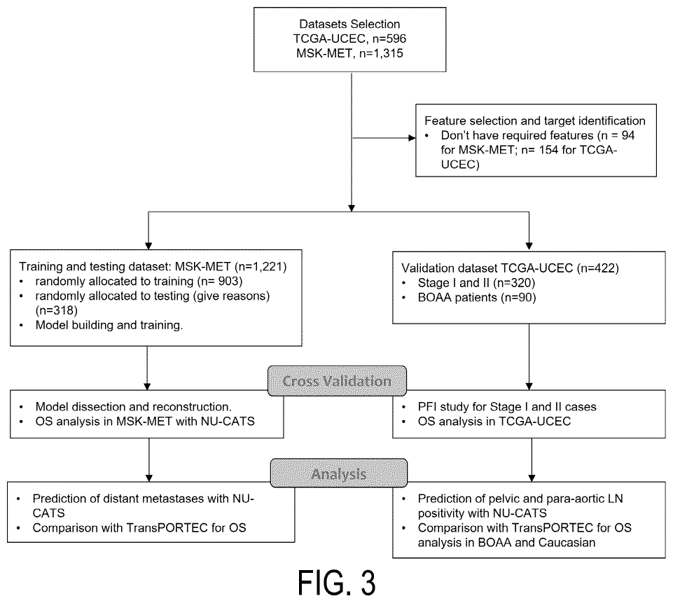

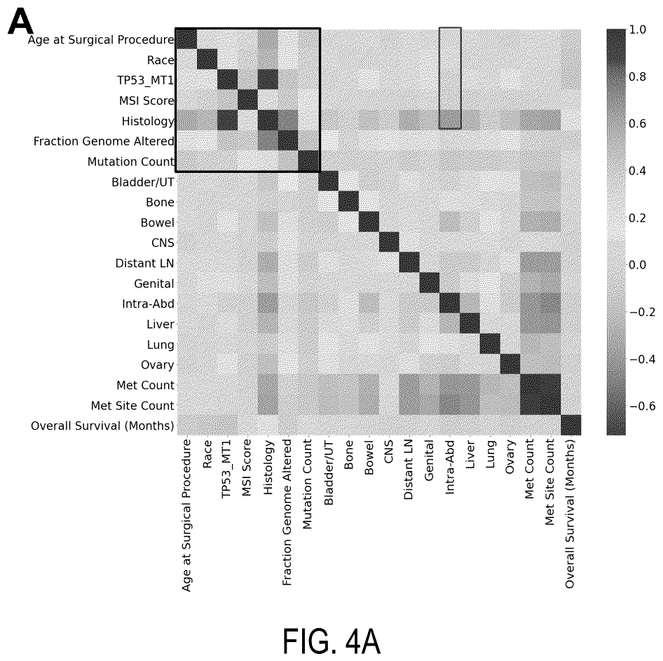

For this example study, endometrial cancer (EC) cases with available clinicopathological and demographic data (age, race, overall survival (OS), and histology subtype), genetic data (TP53, POLE, PTEN and CTNNB1 mutations, MMR status, and copy number variations (CNVs)) and metastasis status were selected from the Memorial Sloan Kettering-Metastatic Events and Tropisms (MSK-MET) dataset (n=1,221). Cross validation of the identified genetic alterations was conducted in the American Association for Cancer Research Project Genomics Evidence Neoplasia Information Exchange (AACR-GENIE v12.0) (n=4,561). Training and testing of the NU-CATS was conducted with the MSK-MET dataset. The Cancer Genome Atlas (TCGA) Uterine Corpus Endometrial Carcinoma (TCGA-UCEC) (n=596) was used as an independent dataset from the MSK-MET to cross-validate the NU-CATS efficacy in predicting local and regional metastasis, OS across different disease stages and races (Black or African American (BOAA) vs. Caucasians) and predicting progression of FIGO Stage I/II diseases. Clinicopathological data for these datasets were accessed via cBioPortal. The CONSORT diagram summarizing case selection for NU-CATS training and validation (FIG. 3).

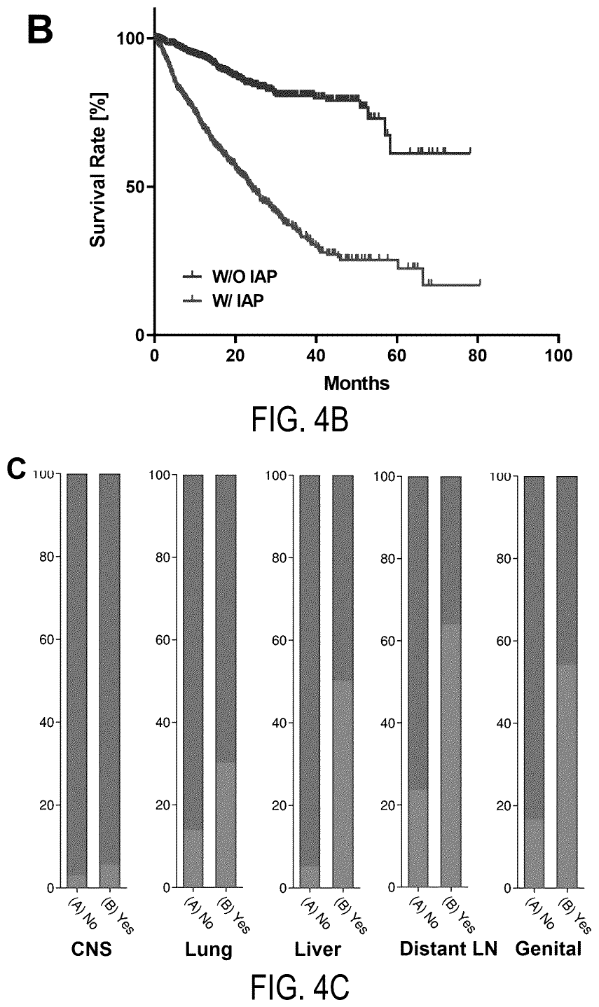

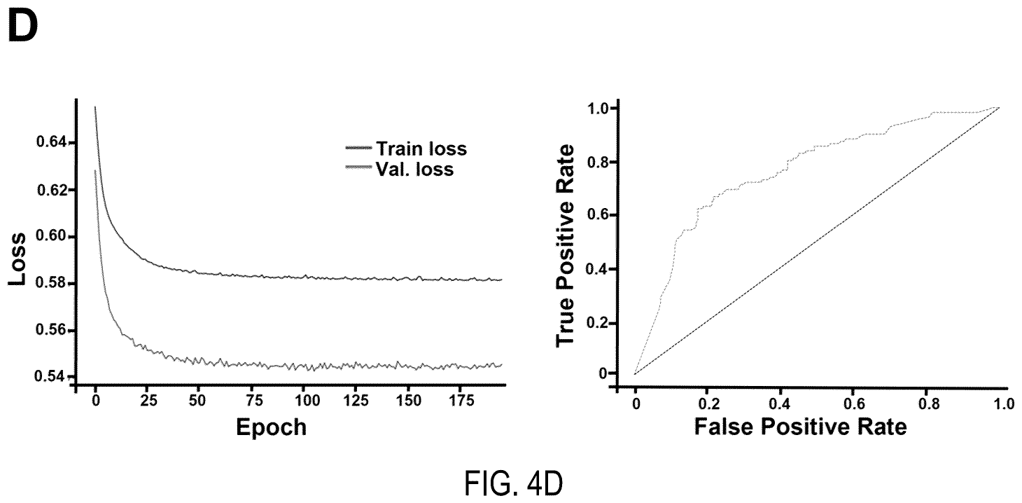

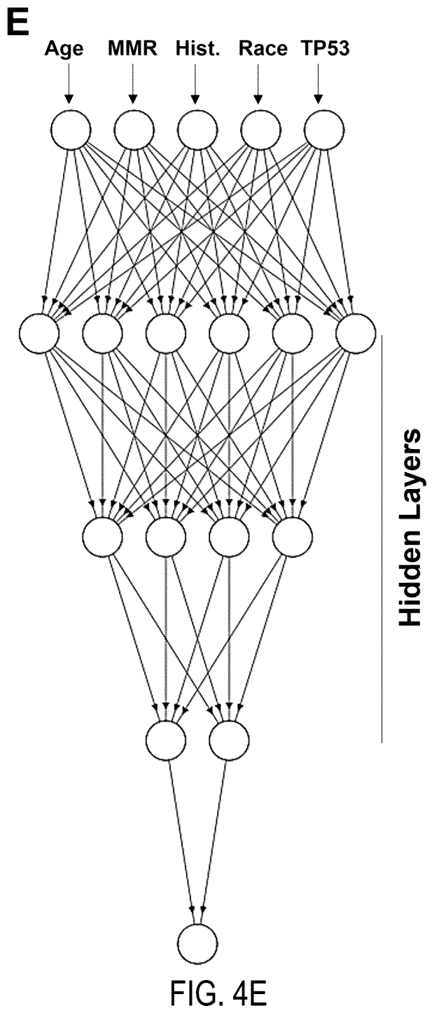

Machine learning can incorporate diverse inputs and, if features are well-selected, yield informed predictions. However, the majority of publicly available datasets lack accurate follow up data on progression free survival (PFS) and overall OS, with the majority of reported outcomes in both TCGA-UCEC and MSK-MET censored. Clinical trial data, by contrast, offer excellent clinical follow up, but typically lack genetic data. Therefore, feature selection and target variable identification for the NU-CATS focus on cost-effective available known prognostic factors, which as a non-limiting example may include age, disease stage, histologic subtype and grades, race, TP53 status, MMR status, fraction genome altered (FGA), and mutation counts. These risk factors were correlated with phenotypes that can represent disease progression as potential target variables including OS, metastases to bladder/urinary tract, bone, liver, lung, bowel, intra-abdominal, central nervous system (CNS), distant lymph node (LN), genital, ovary, as well as the number of both metastatic lesions and metastatic sites (FIG. 4A).

Data preprocessing for MSK-MET and TCGA-UCEC datasets was conducted. The input for ‘Age’ was defined as years of age at surgery or disease diagnosis; histology of endometrioid was represented with ‘1’, and others were assigned ‘0’; genetic abnormalities (i.e., dMMR, TP53, CTNNB1, and PTEN mutations, or CCNE1 amplification) were presented with ‘1’, and wildtype cases were assigned ‘0’; ‘race’ was presented with BOAA patients were assigned with ‘1’, and ‘0’ for non-BOAA patients. For the output, those with intra-abdominal progression (IAP) was assigned with ‘1’, and ‘0’ for those without. In the correlation study, positive metastases were presented with ‘1’ for those with bladder/urinary tract, bone, liver, lung, bowel, intra-abdominal, central nervous system (CNS), distant lymph node (LN), genital or ovary and those without were assigned with ‘0’. The number of metastatic lesions, and metastatic sites, and OS in months were represented with raw data. Correlation studies between genetic and clinicopathological features were conducted with Pandas Dataframe in Python with corr( ) command, and color coded based on correlation coefficient (r) value.

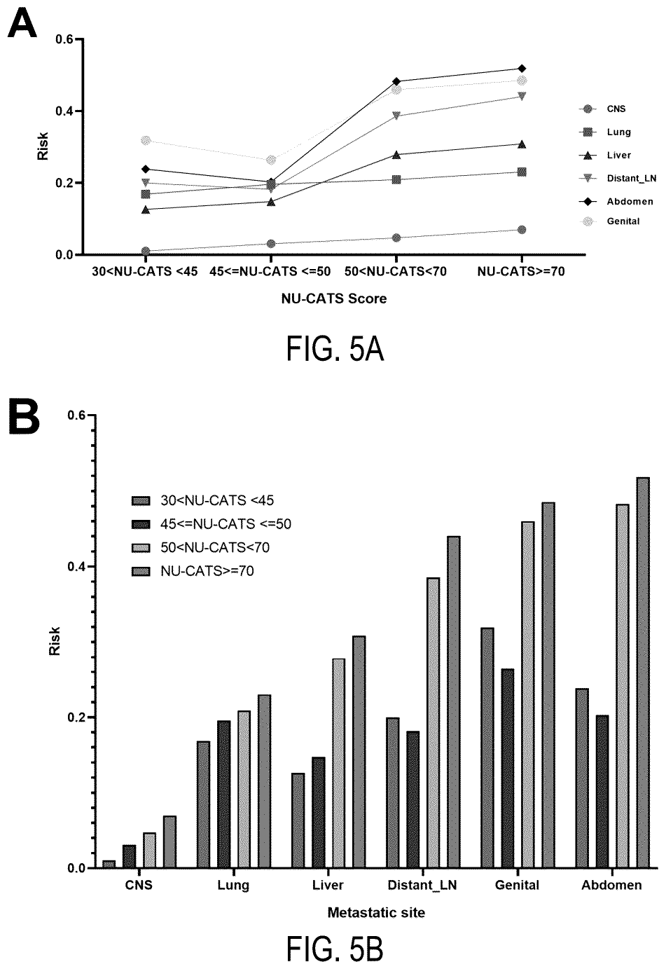

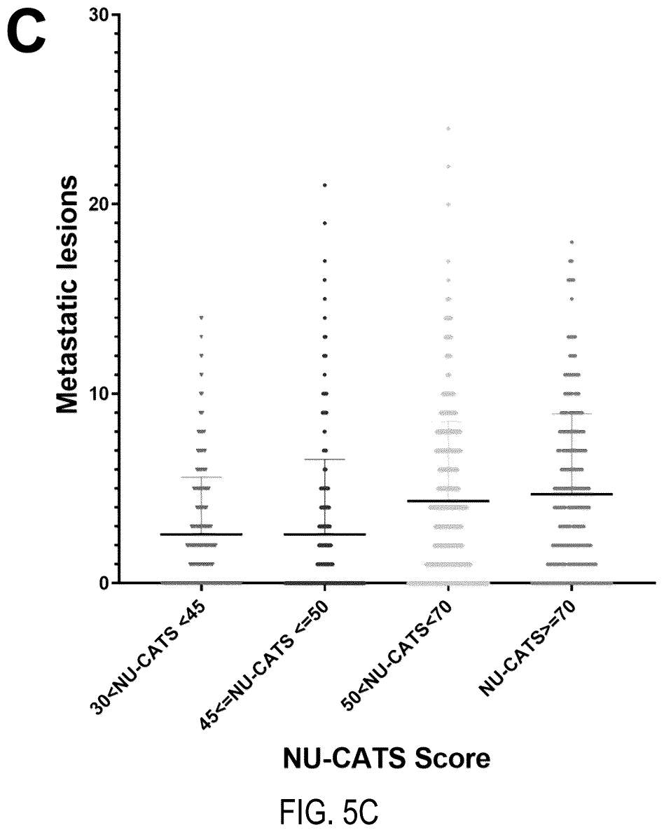

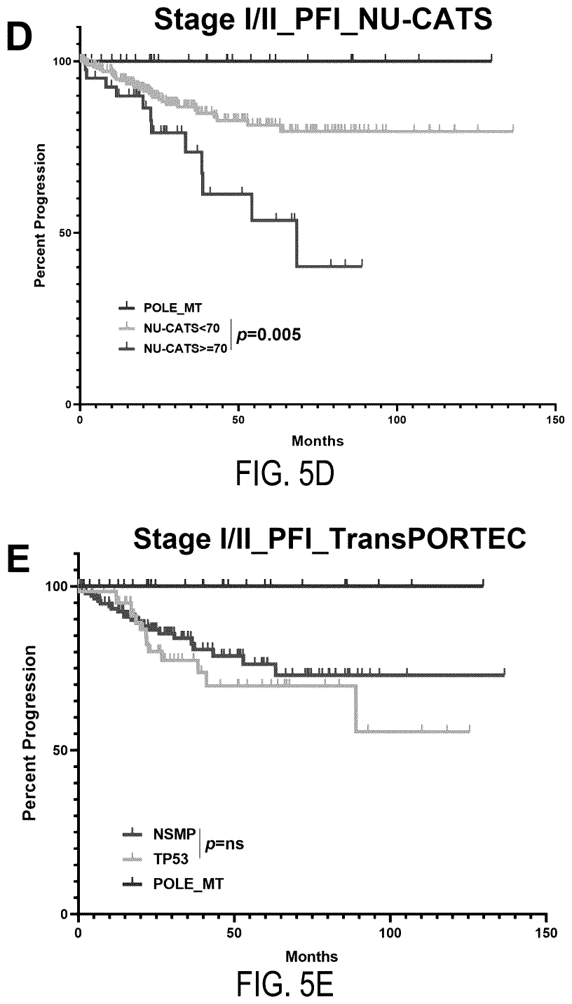

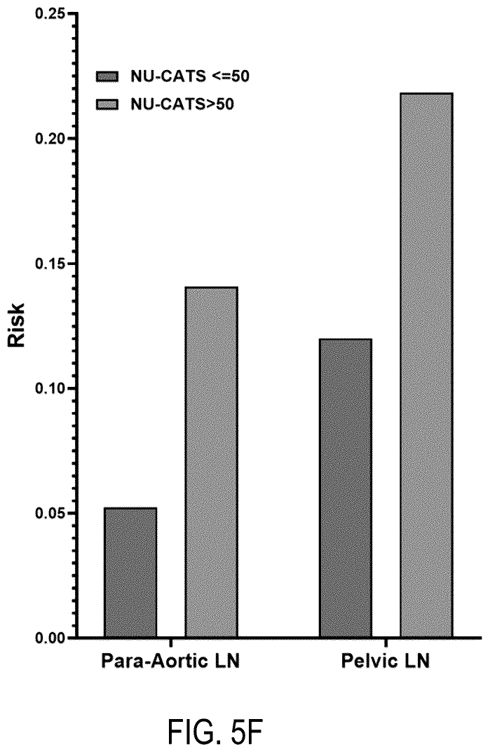

ANNs were constructed using Python. The packages used for creating these neural networks include Keras, Pytorch, and Scikit Learn. After extensive testing, a 5-layer deep learning model (5-6-4-2-1), with a binary output, was built. The first layer contained 5 nodes and received 5 inputs (‘age at surgery’, ‘histology’, ‘race’, ‘mismatch repair status’ and ‘TP53’). The second, third, and fourth layers featured 6, 4, and 2 nodes, respectively, and employed a ReLU activation function. Finally, the output layer was binary with sigmoid activation. Loss was calculated with ‘binary_crossentropy’. FIG. 4E is a schematic overview of the architecture of the example ANN used in the study. The best performing ANN was selected with weights and biases obtained from multiple experiments. The NU-CATS was then calculated for EC patients in the MSK-MET (n=1,221) and TCGA-UCEC (n=442) datasets that have 5 inputs available. The percentages of patients with distant metastases to CNS, lung, liver, bone, distant LN, genital and IAP were calculated based on NU-CATS ranges (30-45, 45-50, 50-70, and ≥70) (FIGS. 5A and 5B).

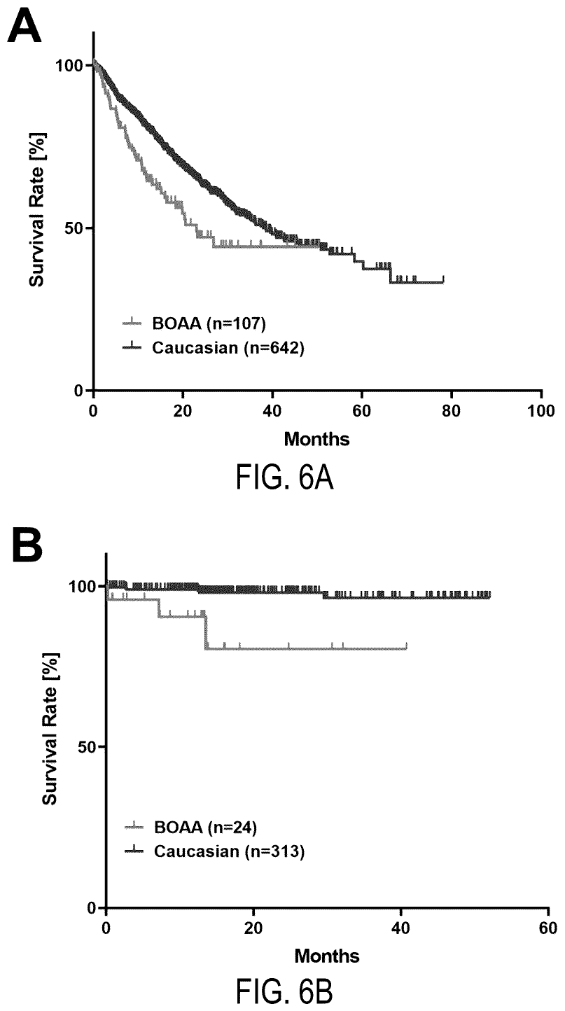

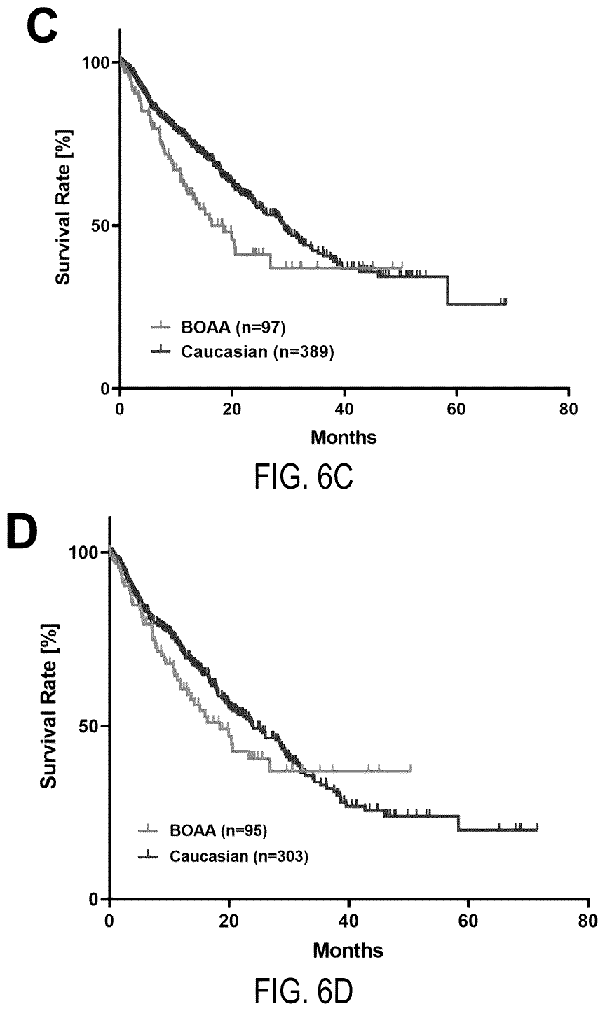

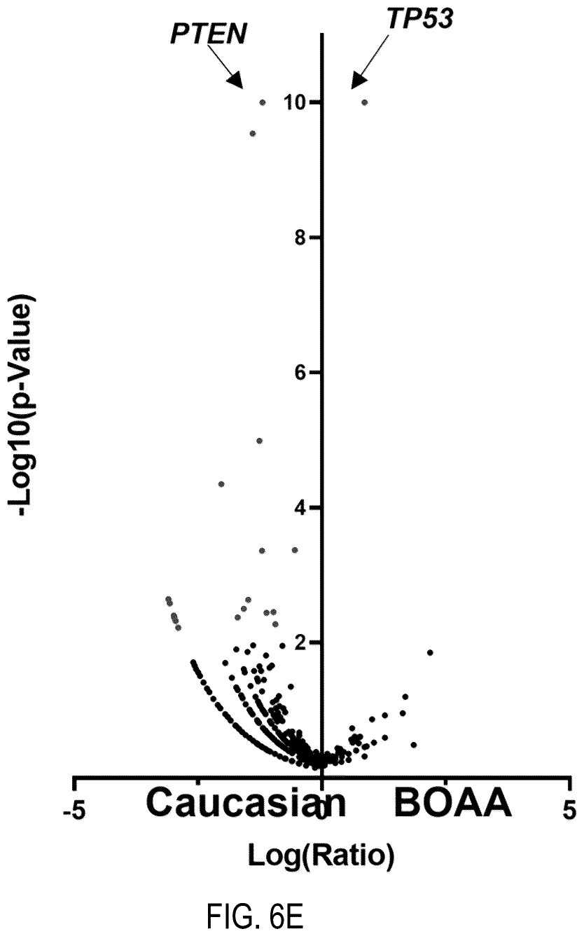

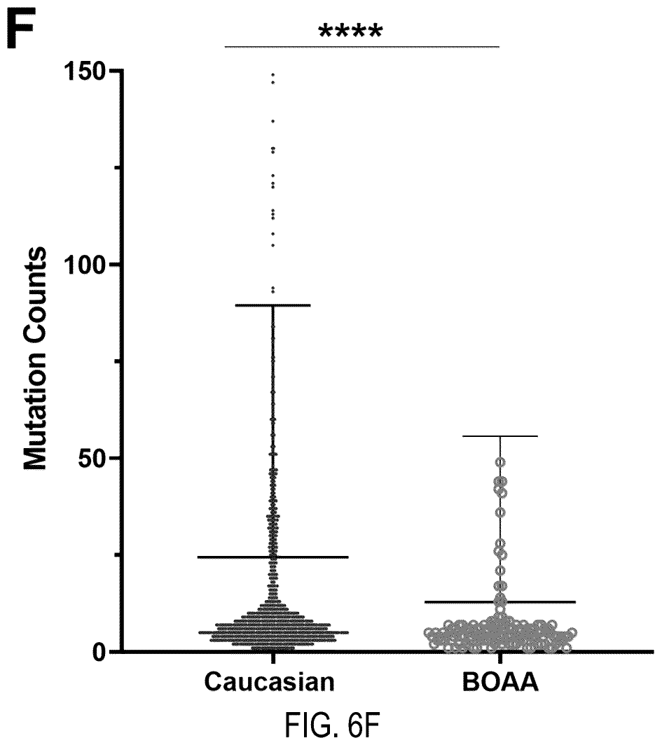

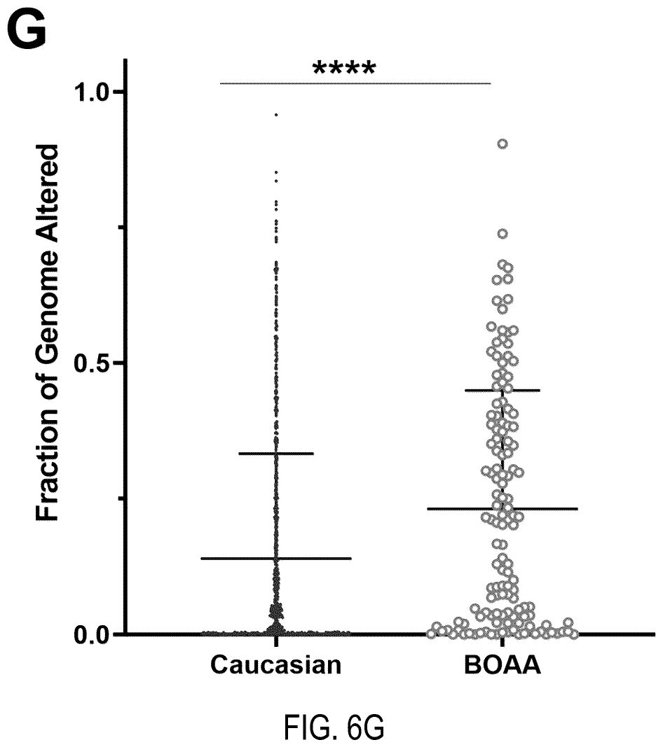

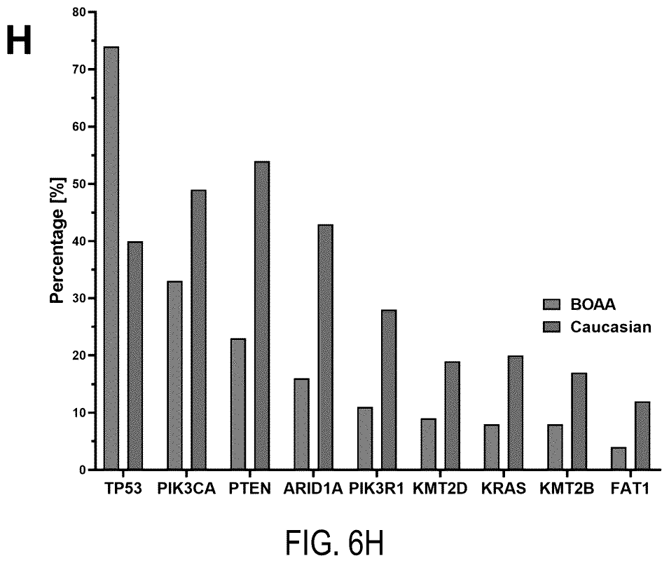

TCGA-UCEC dataset was first studied for possible correlation between race, disease stages, histology subtypes and molecular subgroups. BOAA patients with EC from TCGA-UCEC dataset (n=107) more frequently exhibit the serous histological subtype (30.3% vs. 18.1%), Stage III/IV disease (36.7% vs. 25.6%), and CN-High molecular subtype as compared to Caucasian patients (n=357). Using the MSK-MET data set, which is one of largest dataset with survival and molecular information, overall survival (OS) was compared between BOAA and Caucasian EC patients with or without unfavorable features (metastatic disease, TP53 mutated, uterine serous carcinoma (USC) or carcinosarcoma (UCS) histology). BOAA EC patients have worse OS in the subgroup of EC patients with metastatic lesions (n=107, n=643 for BOAA and Caucasian, respectively, p=0.003), and those without metastatic lesions (n=24, n=313 for BOAA and Caucasian, respectively, p=0.0005), as well as of those with TP53 mutations (n=97, n=389 for BOAA and Caucasian, respectively, p=0.012) (FIGS. 6A-6C). Among patients with UCS or USC histology, no significant survival difference was observed between BOAA (n=95) and Caucasian (n=303) patients (FIG. 6D).

To evaluate this relationship comprehensively, genetic profiling of BOAA (n=415) and Caucasian (n=3,289) EC patients was further carried out using more recent datasets including the AACR-GENIE dataset, in which 71.0% of BOAA patients vs. 41.9% of Caucasian patients carry TP53 mutations (p<0.001). BOAA patients have less frequently exhibit mutations in PTEN, PIK3CA and ARID1A. These results were then cross validated in the MSK-MET dataset (FIGS. 6E, 6H). BOAA EC patients have significantly lower total mutation count, but a higher fraction genome altered (FGA) score (p<0.0001) (FIGS. 6F, 6G). All these results indicate that BOAA EC patients have different genetic alteration profile than Caucasians. Furthermore, only 47.7% of BOAA patients were classified at CN-high subgroup in the TCGA-UCEC dataset, whereas over 70% of BOAA patients carry TP53 mutation using next-generation sequencing (NGS) (FIG. 6H). It is evident that using TP53 as surrogate for CN-high molecular subgroup may imperfectly reflect the unique genetic profile of BOAA EC patients. Therefore, it is reasonable to incorporate race as an independent risk factor in risk classification systems to guide personalized treatment regimens.

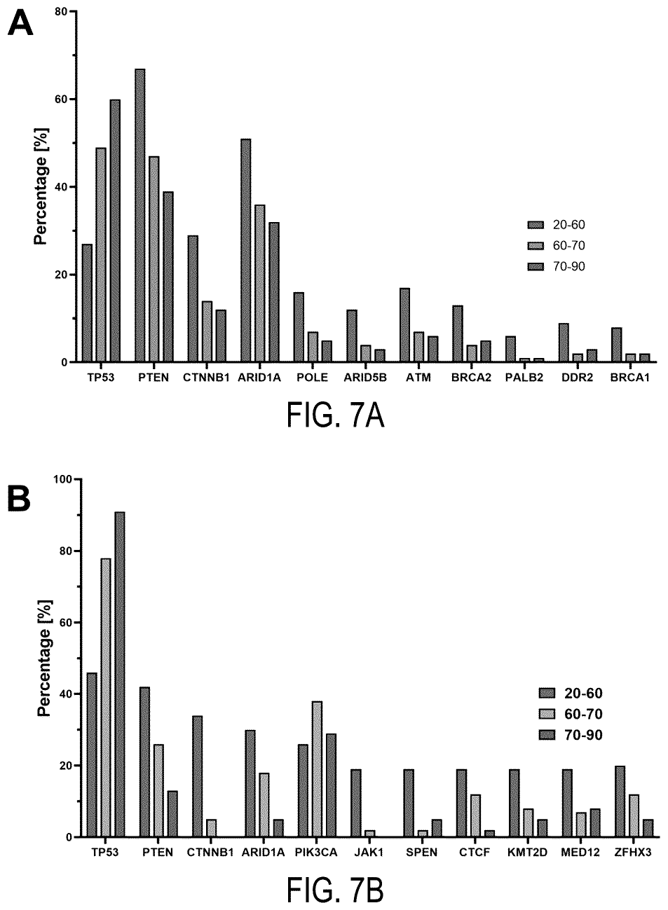

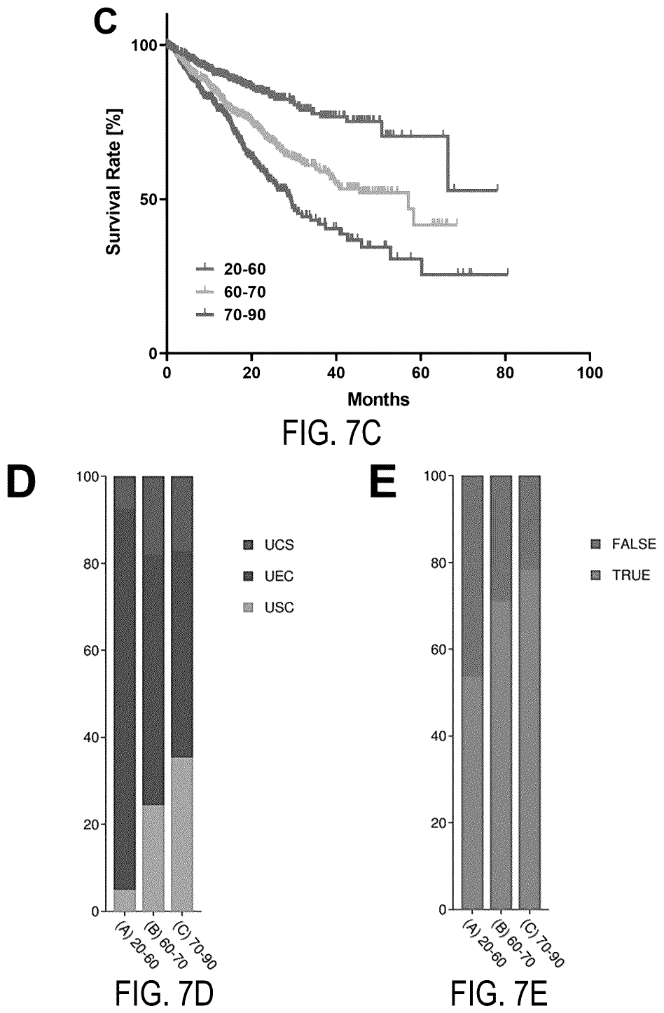

Based on age groups defined by GOG99 and PORTEC1, EC patients in the MSK-MET datasets were subdivided into 3 groups by age between 20 to 60, 60 to 70, and 70 to 90 [14]. The prevalence of TP53 abnormalities in EC patients age 70-90 is more than double that of those age 20-60 (60% vs. 27%, p<0.0001) (FIG. 7A). Meanwhile, genetic alterations related to better prognosis are much more prevalent in younger EC patients with (PTEN: 39% vs. 67%; POLE: 5% vs. 16%, for age 70-90 vs. age 20-60, p<0.0001) (FIG. 7A). In an exception to this rule, CTNNB1 abnormalities are much more prevalent in younger EC patients (12% vs. 29%, for age 70-90 vs. age 20-60, p<0.0001) (FIG. 7A). For BOAA patients, over 90% of EC patients age over 70 have TP53 abnormalities, as compared to 46% for those less than 60 (p<0.001) (FIG. 7B). 34% of BOAA EC patients age 20-60 have CTNNB1 mutation, whereas none of those in the 70-90 age bracket (n=37) expressed this mutation (p<0.0001) (FIG. 7B). Multivariate Cox Regression analysis with risk factors of age, TP53, CTNNB1 and POLE mutation status demonstrated that none of these covariables is significantly correlated with OS while the model incorporating these risk factors are significantly related with survival (p<0.001).

Evaluation of the distribution of histology by age group showed that aging is associated with an increasing percentage of USC subtype (4.9% vs. 35.4%, for age 20-60 vs. age 70-90, p<0.0001) (FIG. 7D). Survival analysis identified that age is significantly associated with poor prognosis (median OS 29.37 vs. 57.4 months, for age 60-70 vs. 70-90, p<0.001) with age 60-70 at intermediate risk (FIG. 7C). It was further found that age is significantly associated risk of having distant metastases (53.73% vs. 71.17% vs. 78.28%, for age 20-60 vs. 60-70 vs. 70-90, p<0.0001) (FIG. 7E). To summarize, while the median age-at-diagnosis of EC patients is in the mid-60s, elder EC patients have an unfavorable genetic background that is associated with less favorable histology and a higher likelihood of developing distant metastases.

The correlation of genetic and clinicopathological features (race, TP53 status, MSI Score, histology, FGA, mutation count) with OS rates was evaluated. The correlation is poor with correlation coefficient (r) ranges between-0.15 to 0.18 (FIG. 4A). The strongest correlations between features and target variables were observed between intra-abdominal progression (IAP) and TP53 mutation (r=0.34), FGA (r=0.32), and histology subtype (r=−0.39) (FIG. 4A). FGA has strong correlation with TP53 (r=0.44) which may biologically a reflection of genomic instability secondary to TP53 mutation (FIG. 4A). Owing to this strong correlation, FGA was excluded from candidate features. TP53 mutation status, histology, race (r=0.1), and MMR (r=−0.05) status constitutes a range of clinicopathological and genetic features (‘5 inputs’ hereafter) that are cost-effective to obtain, and can have positive, intermediate, and negative correlation with IAP (FIG. 4A). IAP was strongly associated with poor survival with patients with (W/) IAP (n=463) and without (W/O) IAP (n=807) has significantly different OS rates (p<0.0001) (FIG. 4B), as well as a higher likelihood of having CNS, distant LN, liver, genital, and lung metastases (FIG. 4C). Therefore, artificial neural network (ANN) models that predict the aggressive phenotype of IAP also likely predict OS and potentially disease progression.

Multiple ANN models were constructed to attempt binary classification. The best performing model with 5 inputs were selected mainly based on receiver operating characteristic (ROC) curve and also avoid overfitting (FIG. 4E). The best performing ANN (5-6-4-2-1) has 5 inputs in the first layer; the second, third and fourth layers are hidden and have 6, 4, and 2 nodes, respectively, with a rectified linear activation function (ReLU) (FIG. 4E). The last layer features binary output with ‘1’ for IAP positive, ‘0’ for IAP negative with a sigmoid activation. The best trained model has area under the curve (AUC) of 0.76 and accuracy of 0.75 in predicting IAP positivity (FIG. 4D). Features such as PTEN, CTNNB1, and POLE mutation status, as well as CCNE1 copy number variations, were also incorporated. The weights and biases were obtained, and the ANN was reconstructed using, as an example, software such as Microsoft Excel®. The scores of the NU-CATS were calculated by taking the integral numbers of sigmoid activation value multiplied by 100.

Among all the metastatic sites (CNS, lung, liver, bone, distant LN, genital and intra-abdominal) studied, elevated NU-CATS≥70 was significantly associated with increased risk of disease progression as compared with those NU-CATS≤50 (p<0.001) (FIGS. 5A, 5B). Elevated NU-CATS are also associated increased total number of metastatic lesions (FIG. 5C). NU-CATS was then cross-validated for predicting disease progression in the TCGA-UCEC dataset. Patients with Stage I/II EC were identified from the TCGA-UCEC dataset and assigned into subgroups with POLE mutation (POLE_MT, n=28), NU-CATS<70 (n=251), NU-CATS≥70 (n=41) (FIG. 5D). The subgroup of NU-CATS≥70 has worse PFS than those less than 70 (p=0.005) (FIG. 5D). No difference in PFS was identified between TP53 group (n=63) and NSMP group (n=141) (p=0.43) when Stage I/II EC patients were classified based on the TransPORTEC classification system (FIG. 5E). By contrast, using NU-CATS in the TCGA-UCEC dataset for cross validated that an elevated NU-CATS score was associated with an increased risk of para-aortic metastases and pelvic LN progression (p=0.0095, 0.0044, respectively) (FIG. 5F). Therefore, NU-CATS can identify a group of EC patients with both higher risks of distant metastases and locoregional progression.

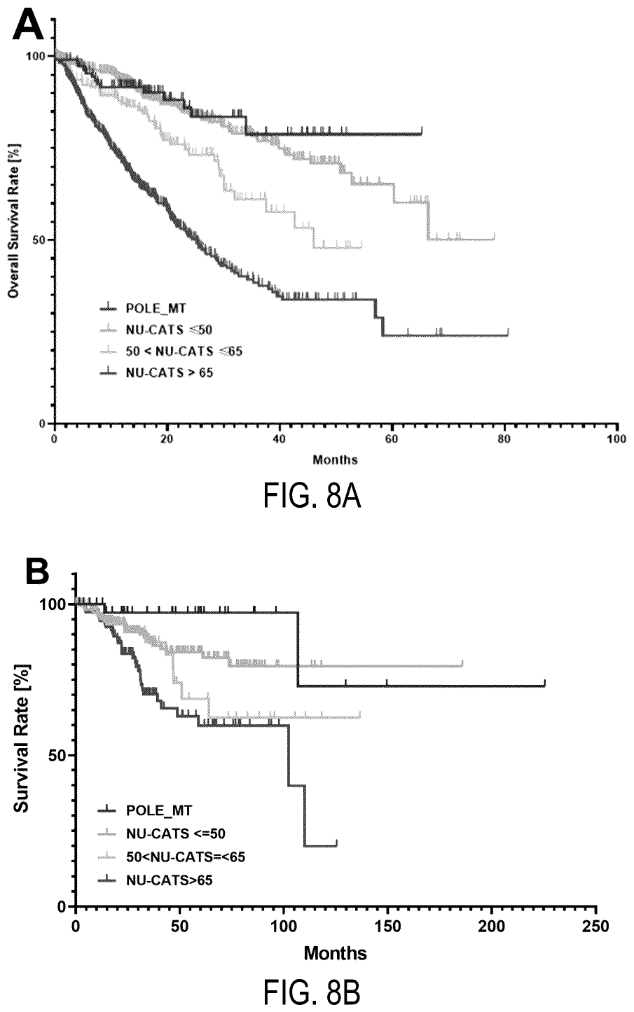

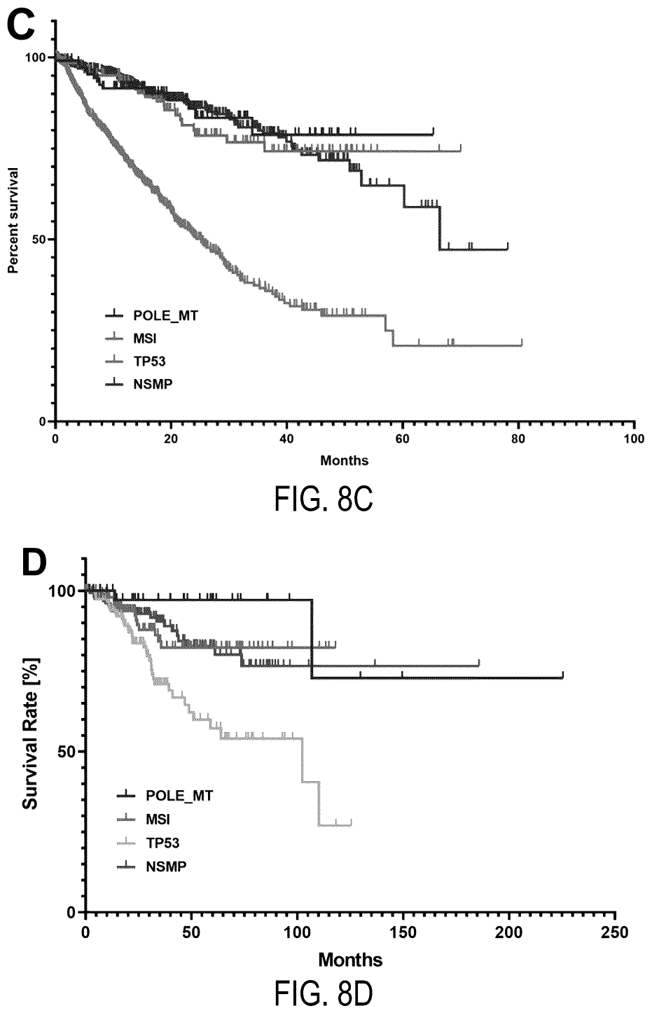

Given the close correlation between disease progression and OS in EC, it is reasonable to hypothesize that NU-CATS is also associated with OS. EC patients from MSK-MET datasets were classified into subgroups of POLE_Mut (n=110), NU-CATS≤50 (n=511), 50<NU-CATS≤65 (n=159), NU-CATS≥65 (n=419). Elevated NU-CATS score was associated with worse OS (p<0.0001) (FIG. 8A). The capability of NU-CATS in prognosticating OS was further cross validated in the TCGA-UCEC dataset with subgroups of POLE_Mut (n=40), NU-CATS≤50 (n=223), 50<NU-CATS≤65 (n=67), NU-CATS≥65 (n=112) (p<0.0001) (FIG. 8B). OS analyses based on TransPORTEC risk classification system were also shown for comparison (FIGS. 8C, 8D). Of note, TransPORTEC system was unable to differentiate NSMP group from MSI group from OS perspective, whereas 4 distinct risk subgroups were identified using NU-CATS (FIG. 8). The NSMP subgroup is the largest subgroup in the TransPORTEC classification system. While subgroups of NU-CATS≥65 and NU-CATS≤50 have similar OS rates as compared to TP53 and NSMP subgroups, respectively, in both MSK-MET and TCGA-UCEC datasets (FIG. 8), NU-CATS identified a group of EC patients with intermediate survival rates with 50<NU-CATS≤65, as shown in both the MSK-MET and TCGA-UCEC datasets (FIGS. 8A, 8B).

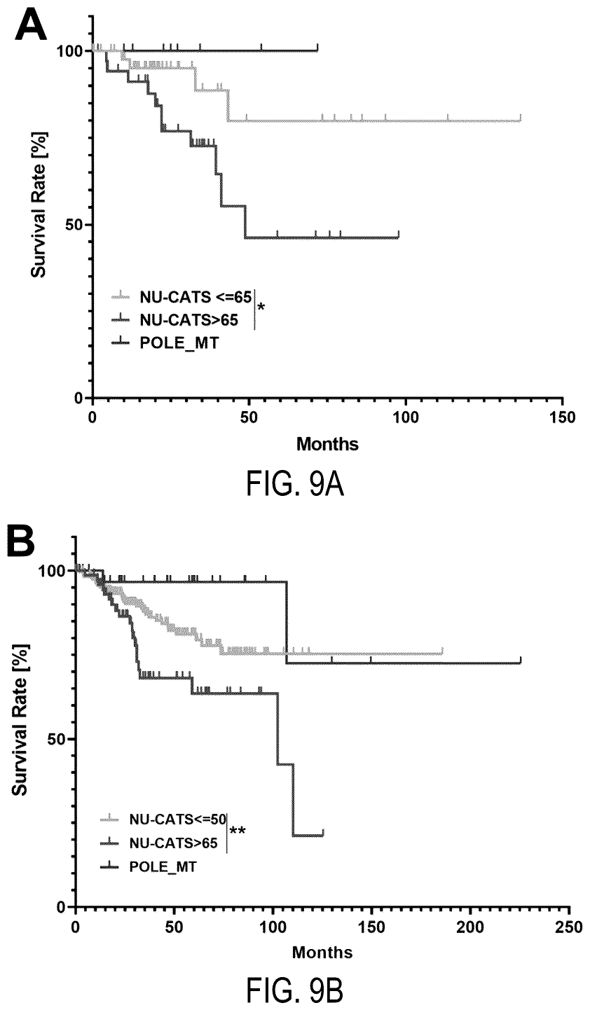

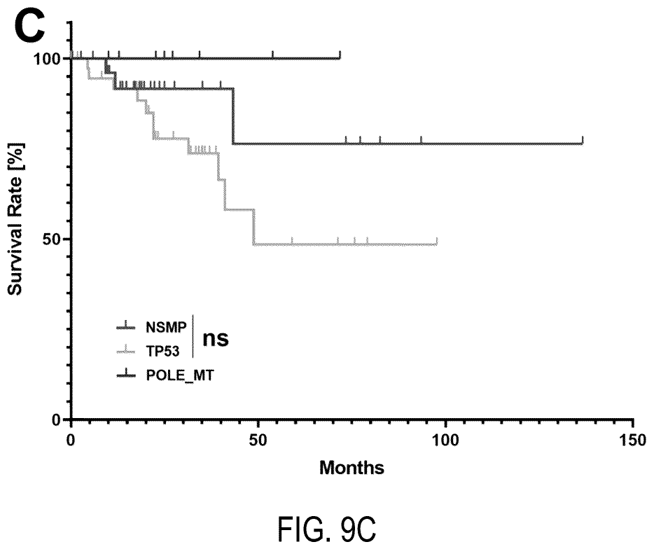

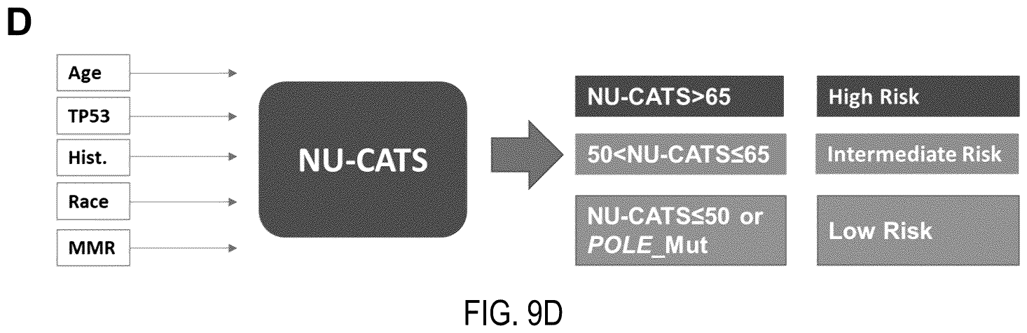

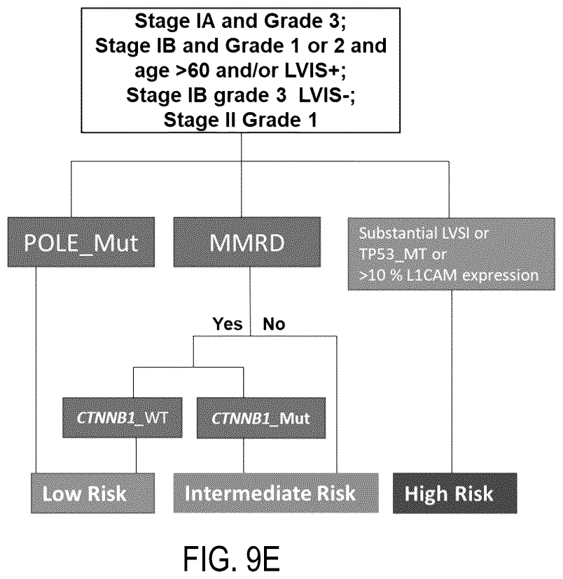

NGS identified TP53 mutations in the majority of BOAA patients (75% in general, over 80% in those age above 60), indicating TransPORTEC system cannot guide personalized therapy since the vast majority of BOAA EC patients will be classified into TP53 positive subgroup (FIG. 7D). The efficacy of NU-CATS and TransPORTEC in risk classification for BOAA EC patients were compared using TCGA-UCEC dataset since it was the dataset originally used to develop molecular classification. BOAA EC patients with elevated NU-CATS>65 (n=36) have a worse prognosis than those with NU-CATS≤65 (n=47) (p=0.03) (FIG. 9A). By contrast, the TransPORTEC system identified no significant OS difference between NSMP (n=30) and TP53 (n=41) of BOAA EC patients in TCGA-UCEC dataset (p=0.16) (FIG. 9C). Of note, the NU-CATS also have good performance in risk classification for Caucasian EC patients elevated NU-CATS>65 (n=75) have worse prognosis than those with NU-CATS≤65 (n=244) (p=0.0097) (FIG. 9B).

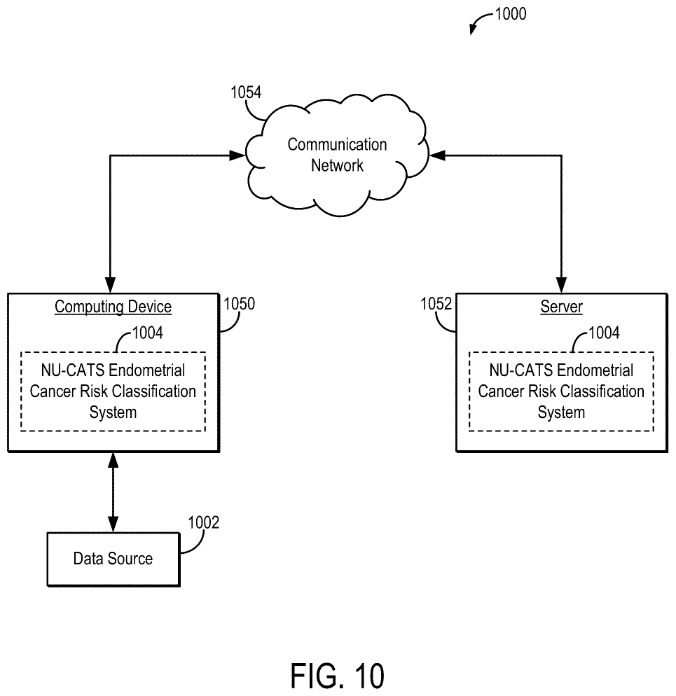

Referring now to FIG. 10, an example of a system 1000 for risk stratifying and/or classifying endometrial cancer in accordance with some embodiments of the systems and methods described in the present disclosure is shown. As shown in FIG. 10, a computing device 1050 can receive one or more types of data (e.g., patient health data) from data source 1002. In some embodiments, computing device 1050 can execute at least a portion of a NU-CATS endometrial cancer risk classification system 1004 to generate classified feature data from patient health data received from the data source 1002.

Additionally or alternatively, in some embodiments, the computing device 1050 can communicate information about data received from the data source 1002 to a server 1052 over a communication network 1054, which can execute at least a portion of the NU-CATS endometrial cancer risk classification system 1004. In such embodiments, the server 1052 can return information to the computing device 1050 (and/or any other suitable computing device) indicative of an output of the NU-CATS endometrial cancer risk classification system 1004.

In some embodiments, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. The computing device 1050 and/or server 1052 can also reconstruct images from the data.

In some embodiments, data source 1002 can be any suitable source of data (e.g., patient health data), another computing device (e.g., a server storing patient health data), and so on. In some embodiments, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some embodiments, data source 1002 can be located locally and/or remotely from computing device 1050, and can communicate data to computing device 1050 (and/or server 1052) via a communication network (e.g., communication network 1054).

In some embodiments, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1054 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, and so on.

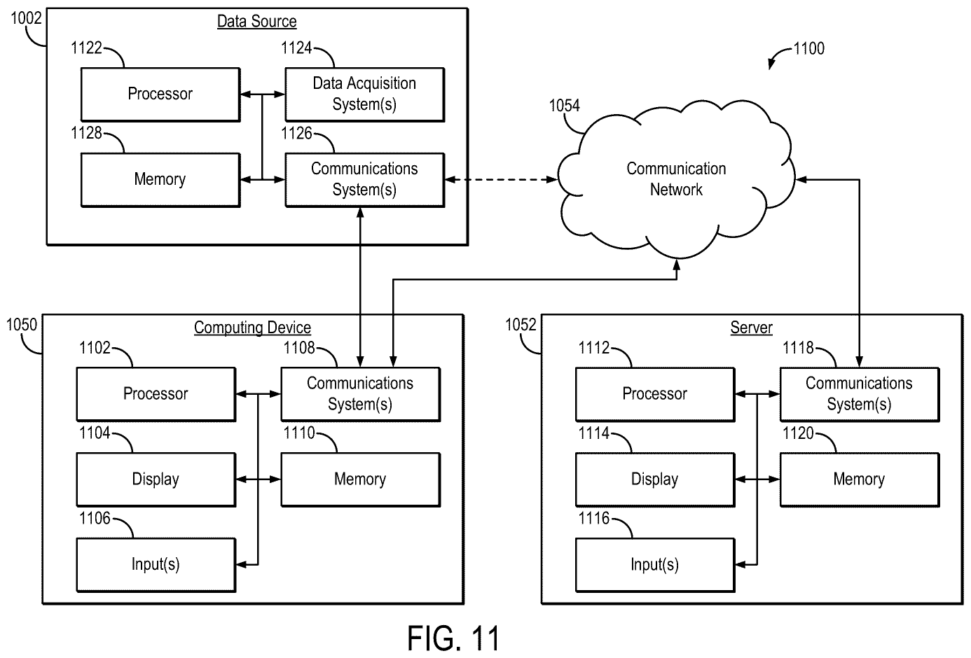

Referring now to FIG. 11, an example of hardware 1100 that can be used to implement data source 1002, computing device 1050, and server 1052 in accordance with some embodiments of the systems and methods described in the present disclosure is shown.

As shown in FIG. 11, in some embodiments, computing device 1050 can include a processor 1102, a display 1104, one or more inputs 1106, one or more communication systems 1108, and/or memory 1110. In some embodiments, processor 1102 can be any suitable hardware processor or combination of processors, such as a central processing unit (“CPU”), a graphics processing unit (“GPU”), and so on. In some embodiments, display 1104 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1106 can include any suitable input devices and/or sensors that can be used to receive user input, such as a keyboard, a mouse, a touchscreen, a microphone, and so on.

In some embodiments, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some embodiments, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1110 can have encoded thereon, or otherwise stored therein, a computer program for controlling operation of computing device 1050. In such embodiments, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein (e.g., the method of FIG. 1, the method of FIG. 2).

In some embodiments, server 1052 can include a processor 1112, a display 1114, one or more inputs 1116, one or more communications systems 1118, and/or memory 1120. In some embodiments, processor 1112 can be any suitable hardware processor or combination of processors, such as a CPU, a GPU, and so on. In some embodiments, display 1114 can include any suitable display devices, such as an LCD screen, LED display, OLED display, electrophoretic display, a computer monitor, a touchscreen, a television, and so on. In some embodiments, inputs 1116 can include any suitable input devices and/or sensors that can be used to receive user input, such as a keyboard, a mouse, a touchscreen, a microphone, and so on.

In some embodiments, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some embodiments, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1120 can have encoded thereon a server program for controlling operation of server 1052. In such embodiments, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some embodiments, the server 1052 is configured to perform the methods described in the present disclosure. For example, the processor 1112 and memory 1120 can be configured to perform the methods described herein (e.g., the method of FIG. 1, the method of FIG. 2).

In some embodiments, data source 1002 can include a processor 1122, one or more data acquisition systems 1124, one or more communications systems 1126, and/or memory 1128. In some embodiments, processor 1122 can be any suitable hardware processor or combination of processors, such as a CPU, a GPU, and so on. In some embodiments, the one or more data acquisition systems 1124 are generally configured to acquire data, images, or both. Additionally or alternatively, in some embodiments, the one or more data acquisition systems 1124 can include any suitable hardware, firmware, and/or software for coupling to and/or controlling operations of a data acquisition system. In some embodiments, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some embodiments, communications systems 1126 can include any suitable hardware, firmware, and/or software for communicating information to computing device 1050 (and, in some embodiments, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1128 can have encoded thereon, or otherwise stored therein, a program for controlling operation of data source 1002. In such embodiments, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for risk stratifying a patient for endometrial cancer using machine learning, comprising:
accessing patient health data for a patient with a computer system;
accessing a machine learning model with the computer system, wherein the machine learning model has been trained on training data to generate classified feature data based on features present in a patient's patient health data;
applying the patient health data to the machine learning model, generating an output as classified feature data that indicate at least one of a risk stratification or classification of endometrial cancer in the patient based on features in their patient health data; and
outputting the classified feature data with the computer system.

2. The method of claim 1, wherein the machine learning model comprises an artificial neural network.

3. The method of claim 2, wherein the artificial neural network is a deep neural network.

4. The method of claim 1, further comprising selecting a subset of features from the patient health data and inputting only the subset of features to the machine learning model.

5. The method of claim 4, wherein the subset of features is determined by training another machine learning model on patient health data collected from a cohort of patients.

6. The method of claim 4, wherein the subset of features comprises patient demographic data and molecular data.

7. The method of claim 6, wherein the patient demographic data comprises age and race.

8. The method of claim 6, wherein the molecular data comprise at least one of TP53 status, mismatch repair (MMR) status, fraction genome altered (FGA), and mutation counts.

9. The method of claim 6, wherein the subset of features further comprises at least one of histologic subtype or histologic grade.

10. The method of claim 1, wherein the classified feature data comprise probability values for developing endometrial cancer.

11. The method of claim 1, wherein the classified feature data comprise category labels indicating low, moderate, or high risk for developing endometrial cancer.

12. The method of claim 1, wherein outputting the classified feature data comprises:
analyzing the classified feature data with the computer system to determine a risk for the patient developing endometrial cancer;
generating an order set for a follow up examination of the patient based on the determined risk for the patient developing endometrial cancer; and
outputting the order set to an electronic health record (EHR) of the patient using the computer system.

13. The method of claim 12, wherein the determined risk for the patient developing endometrial cancer indicates a risk of nodal involvement for endometrial cancer in the patient and the order set indicates orders for examination to determine an extent of nodal involvement for endometrial cancer in the patient.

14. The method of claim 12, wherein the order set indicates a treatment option for the patient based on the determined risk for the patient developing endometrial cancer.
